(19) United States
(12) Patent Application Publication
(10) Pub. No.: US 2023/0172924 A1
(43) Pub. Date: Jun. 8, 2023

(54) THERAPEUTIC AGENT FOR BREAST CANCER

(71) Applicant: Eisai R&D Management Co., Ltd., Tokyo (JP)

(72) Inventors: Satoshi KAWANO, Tsukuba (JP); Saori Miyano, Tsukubamirai (JP); Kyoko NISHIBATA, Tsukuba (JP); Sayo FUKUSHIMA, Tsukuba (JP)

(21) Appl. No.: 18/008,304

(22) PCT Filed: Jul. 29, 2021
(86) PCT No.: PCT/JP2021/028008

§ 371 Date: Dec. 5, 2022

(30) Foreign Application Priority Data
Jul. 31, 2020 (JP) 2020130935
Sep. 29, 2020 (JP) 2020163258

(51) Int. Cl. A61K 31/4545; A61K 31/565; A61K 31/4196; A61K 31/566; A61P 35/00

(52) U.S. Cl. CPC A61K 31/4545; A61K 31/565; A61K 31/4196; A61K 31/566; A61P 35/00

(57) ABSTRACT

Disclosed is a therapeutic agent for breast cancer, said therapeutic agent comprising 5-((2-(4-(1-(2-hydroxyethyl)piperidin-4-yl)benzamide)pyridin-4-yl)oxy)-6-(2-methoxyethoxy)-N-methyl-1H-indole-1-carboxamide or a pharmacologically acceptable salt thereof that is to be administered in combination with an estrogen receptor antagonist or an aromatase inhibitor.

### TECHNICAL FIELD

The present invention relates to a therapeutic agent for breast cancer in which a monocyclic pyridine derivative having fibroblast growth factor receptor (FGFR) inhibitory action or a pharmacologically acceptable salt thereof; and an estrogen receptor antagonist or an aromatase inhibitor are used in combination. More specifically, the present invention relates to a therapeutic agent for breast cancer administered in combination with an estrogen receptor antagonist or an aromatase inhibitor, wherein the therapeutic agent for breast cancer comprises 5-((2-(4-(1-(2-hydroxyethyl)piperidin-4-yl)benzamide)pyridin-4-yl)oxy)-6-(2-methoxyethoxy)-N-methyl-11H-indole-1-carboxamide or a pharmacologically acceptable salt thereof.

### BACKGROUND ART

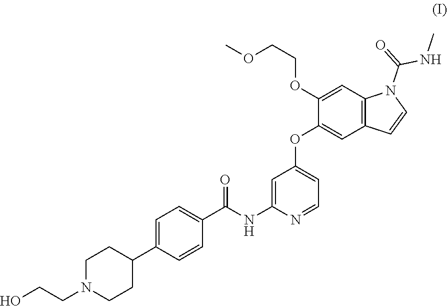 [SMILES: CNC(=O)n1ccc2cc(Oc3ccnc(NC(=O)c4ccc(C5CCN(CCO)CC5)cc4)c3)c(OCCOC)cc21]

5-((2-(4-(1-(2-Hydroxyethyl)piperidin-4-yl)benzamide)pyridin-4-yl)oxy)-6-(2-methoxyethoxy)-N-methyl-1H-indole-1-carboxamide represented by Formula (I) is known as an inhibitor against FGFR1, FGFR2, and FGFR3, and it is reported that the above compound has inhibitory action on cell proliferation of stomach cancer, lung cancer, bladder cancer, and endometrial cancer (Patent Literature 1). It is reported that the above compound exerts a high therapeutic effect against bile duct cancer (Patent Literature 2), breast cancer (Patent Literature 3), and hepatocellular carcinoma (Patent Literature 4). As a pharmacologically acceptable salt of the above compound, succinate and maleate are known (Patent Literature 5).

Estrogen receptor antagonists such as tamoxifen and fulvestrant are used for treating estrogen receptor-positive breast cancer (Non Patent Literature 1).

Aromatase inhibitors such as exemestane and anastrozole inhibit aromatase which converts androgen secreted from the adrenal cortex into estrogen (Non Patent Literature 2). For this reason, they are used for treating estrogen receptor-positive breast cancer similarly to the estrogen receptor antagonists. In general, aromatase inhibitors are used in postmenopausal breast cancer patients, whereas estrogen receptor antagonists are used in premenopausal breast cancer patients.

Breast cancer is classified based on the presence or absence of expression of estrogen receptor, progesterone receptor, and human epidermal growth factor receptor type 2 (HER2), and, together with surgical removal of a lesion part, drug therapy is performed depending on the classification.

### CITATION LIST

### Patent Literature

[Patent Literature 1] US Patent Publication No. 2014-0235614
[Patent Literature 2] US Patent Publication No. 2018-0015079
[Patent Literature 3] US Patent Publication No. 2018-0303817
[Patent Literature 4] WO 2019/189241
[Patent Literature 5] US Patent Publication No. 2017-0217935

### Non-Patent Literature

[Non Patent Literature 1] Howell et al., “Comparison of Fulvestrant Versus Tamoxifen for the Treatment of Advanced Breast Cancer in Postmenopausal Women Previously Untreated With Endocrine Therapy: A Multinational, Double-Blind, Randomized Trial”, Journal of Clinical Oncology, 2004, 22(9), 1605-1613.
[Non Patent Literature 2] Deeks et al., “Exemestane A Review of its Use in Postmenopausal Women with Breast Cancer” Drugs, 2009, 69(7), 889-918

### SUMMARY OF INVENTION

### Technical Problem

An object of the present invention is to provide a therapeutic agent for breast cancer which involves combinational administration of a plurality of medicines.

### Solution to Problem

In view of such circumstances, the present inventors have conducted extensive studies, and, as a result, they have found that administration of a combination of the above-described compound represented by Formula (I) and an estrogen antagonist or an aromatase inhibitor exerts a high therapeutic effect against breast cancer, thus leading to realization of the present invention.

That is, the present invention provides [1] to [18] below.

[1] A therapeutic agent for breast cancer, comprising: 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I):

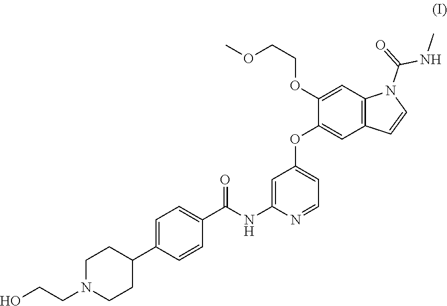 [SMILES: CNC(=O)n1ccc2cc(Oc3ccnc(NC(=O)c4ccc(C5CCN(CCO)CC5)cc4)c3)c(OCCOC)cc21]

or a pharmacologically acceptable salt thereof which is administered in combination with an estrogen receptor antagonist or an aromatase inhibitor.
[2] A pharmaceutical composition for treating breast cancer, comprising: 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof which is administered in combination with an estrogen receptor antagonist or an aromatase inhibitor.
[3] A pharmaceutical composition for treating breast cancer, comprising: 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof, and an estrogen receptor antagonist or an aromatase inhibitor.
[4] A kit for treating breast cancer, comprising: a preparation comprising 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof, and a preparation comprising an estrogen receptor antagonist or an aromatase inhibitor.
[5] A method for treating breast cancer, comprising: administering 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof, and an estrogen receptor antagonist or an aromatase inhibitor to a patient in need thereof.
[6] 5-((2-(4-(1-(2-Hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof for use in treatment of breast cancer, which is administered in combination with an estrogen receptor antagonist or an aromatase inhibitor.
[7] A combination for treating breast cancer, comprising: 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof; and an estrogen receptor antagonist or an aromatase inhibitor.
[8] Use of 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof, which is administered in combination with an estrogen receptor antagonist or an aromatase inhibitor, for the manufacture of a therapeutic agent for breast cancer.
[9] The therapeutic agent, composition, kit, method, compound, combination, or use, wherein 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof, and the estrogen receptor antagonist or the aromatase inhibitor are administered simultaneously or separately.
[10] The therapeutic agent, composition, kit, method, compound, combination, or use, wherein the pharmacologically acceptable salt is 1.5 succinate.
[11] The therapeutic agent, composition, kit, method, compound, combination, or use, wherein the estrogen receptor antagonist is fulvestrant, tamoxifen or a pharmacologically acceptable salt thereof, or mepitiostane.
[12] The therapeutic agent, composition, kit, method, compound, combination, or use, wherein the estrogen receptor antagonist is fulvestrant.
[13] The therapeutic agent, composition, kit, method, compound, combination, or use, wherein the aromatase inhibitor is exemestane, anastrozole, or letrozole.
[14] The therapeutic agent, composition, kit, method, compound, combination, or use, wherein the aromatase inhibitor is exemestane.
[14a] The therapeutic agent, composition, kit, method, compound, combination, or use, wherein the aromatase inhibitor is letrozole.
[15] The therapeutic agent, composition, kit, method, compound, combination, or use, wherein the breast cancer is estrogen receptor-positive.
[16] The therapeutic agent, composition, kit, method, compound, combination, or use, wherein the breast cancer is locally advanced breast cancer, metastatic breast cancer, recurrent breast cancer, or unresectable breast cancer.
[17] The therapeutic agent, composition, kit, method, compound, combination, or use, which is for use in treatment of breast cancer fibroblast growth factor receptor (FGFR).
[18] The therapeutic agent, composition, kit, method, compound, combination, or use, wherein FGFR is FGFR1, FGFR2, or FGFR3.

### Advantageous Effects of Invention

By administering a combination of the compound represented by Formula (I) and an estrogen antagonist or an aromatase inhibitor, there is a probability that an effect of reducing a tumor volume against breast cancer may be exhibited.

### BRIEF DESCRIPTION OF DRAWINGS

FIG. 1 is a graph showing changes in average tumor volume in each group after starting drug administration in Example 1.

FIG. 2 is a graph showing changes in average tumor volume in each group after starting drug administration in Example 4.

### DESCRIPTION OF EMBODIMENTS

A compound represented by Formula (I) or a pharmacologically acceptable salt thereof according to the present invention can be produced through a method disclosed in Patent Literature 1.

In the present specification, examples of pharmacologically acceptable salts include a salt with an inorganic acid, a salt with an organic acid, and a salt with an acidic amino acid.

Examples of suitable examples of salts with inorganic acids include salts with hydrochloric acid, hydrobromic acid, sulfuric acid, nitric acid, and phosphoric acid.

Suitable examples of salts with organic acids include salts with acetic acid, succinic acid, fumaric acid, maleic acid, tartaric acid, citric acid, lactic acid, stearic acid, benzoic acid, methanesulfonic acid, ethanesulfonic acid, and p-toluenesulfonic acid.

Examples of suitable examples of salts with acid amino acids include salts with aspartic acid and glutamic acid.

A preferred pharmacologically acceptable salt is succinate or maleate, and a more preferred salt is succinate. 1.5 Succinate is particularly preferable (hereinafter, 1.5 succinate of the compound represented by Formula (I) is denoted as a compound A).

The therapeutic agent for breast cancer according to the present invention can be administered orally in a form of a solid preparation such as a tablet, granules, fine granules, powders, or a capsule, a liquid, a jelly, a syrup, or the like. In addition, the therapeutic agent for breast cancer according to the present invention may be administered parenterally in a form of an injection, a suppository, an ointment, a poultice, or the like.

The therapeutic agent for breast cancer according to the present invention can be formulated by the methods described in the Japanese Pharmacopoeia (JP), the European Pharmacopoeia (EP), or the United States Pharmacopeia (USP).

The dose of the compound represented by Formula (I) or a pharmacologically acceptable salt thereof can be appropriately selected depending on the severity of symptoms, the age, sex, body weight, and differential sensitivity of a patient, the administration method, the administration period, the administration intervals, the dosage form, and the like. In a case of oral administration to an adult (body weight of 60 kg), the dose is 0.5 mg to 5 g, preferably 1 mg to 1 g, and furthermore preferably 1 mg to 500 mg per day. This dose can be administered in 1 to 3 divided portions per day.

In the present specification, the estrogen receptor antagonist means a medicine that binds to an estrogen receptor expressed in breast cancer cells. By the mechanism, the estrogen receptor antagonist can inhibit the binding of estrogen receptors to estrogen and suppress the proliferation of breast cancer cells.

Suitable examples of estrogen receptor antagonists include fulvestrant, tamoxifen or a pharmacologically acceptable salt thereof (such as citrate), and mepitiostane. Fulvestrant is preferable.

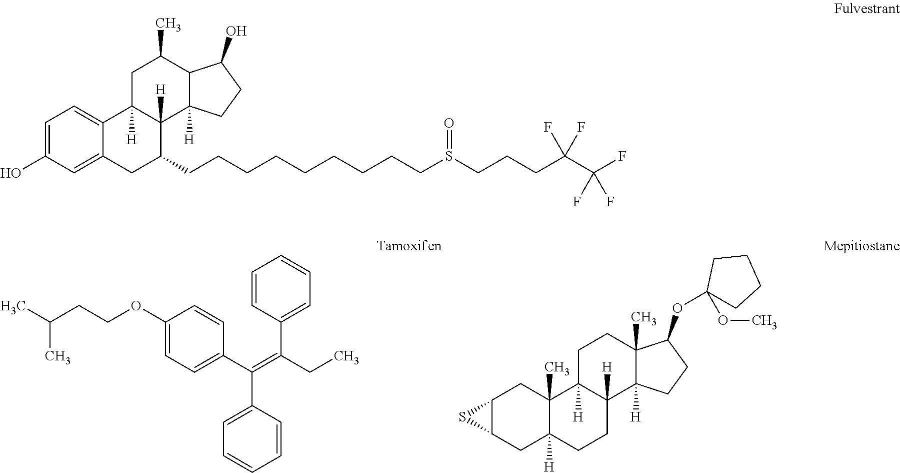

In addition, the estrogen receptor antagonist can be elacestrant, H3B-6545, toremifene citrate, SAR439859, AZD9833, rintodestrant, ZN-c5, LSZ102, D-0502, LY3484356, SHR9549, brilanestrant, and giredestrant.

Regarding the dose and administration method of the estrogen receptor antagonist, in a case where, for example, the estrogen receptor antagonist is fulvestrant, 500 mg thereof is administered intramuscularly per dose at an initial dose, 2 weeks later, 4 weeks later, and every 4 weeks thereafter.

In a case where the estrogen receptor antagonist is tamoxifen or a pharmacologically acceptable salt thereof, 20 to 40 mg (as tamoxifen) per day is administered orally in 1 to 2 divided doses.

In a case where the estrogen receptor antagonist is mepitiostane, 20 mg per day is administered orally in 2 divided doses.

In addition, an aromatase inhibitor that inhibits synthesis of estrogen may be used instead of the estrogen receptor antagonist. Suitable examples of aromatase inhibitors include exemestane, anastrozole, and letrozole. Exemestane is preferable.

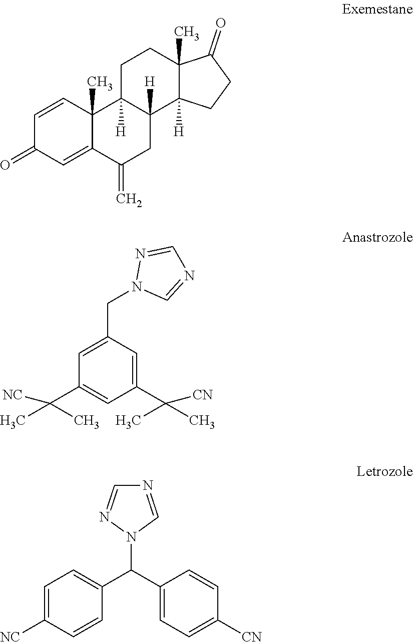

Regarding the dose and administration route of the aromatase inhibitor, in a case where, for example, the aromatase inhibitor is exemestane, 25 mg thereof is administered orally once a day after meals.

In a case where the aromatase inhibitor is anastrozole, 1 mg thereof is administered orally once a day.

In a case where the aromatase inhibitor is letrozole, 2.5 mg thereof is administered orally once a day.

In the present specification, administrating in combination means that a preparation comprising the compound represented by Formula (I) or a pharmacologically acceptable salt thereof; and a preparation comprising an estrogen receptor antagonist or an aromatase inhibitor are administered to a patient simultaneously or separately. In addition, a composition comprising the compound represented by Formula (I) or a pharmacologically acceptable salt thereof; and an estrogen receptor antagonist or an aromatase inhibitor in one preparation may be administered.

In the present specification, breast cancer means a benign or malignant tumor developed in mammary glands (milk ducts and lobules). The breast cancer includes locally advanced breast cancer, metastatic breast cancer, recurrent breast cancer, or unresectable breast cancer.

### EXAMPLES

The present invention will be described in more detail with reference to the following examples.

Example 1 Proliferation inhibitory action against human breast cancer patient-derived tumor (OD-BRE-0438) due to combined use of compound A and fulvestrant (trade name: Faslodex intramuscular injection 250 mg, AstraZeneca PLC)

Five NOD-SCID mice (NOD. CB17-Prkdcscid/J, female, Charles River Laboratories Japan, Inc.) were used in each group to evaluate antitumor effect in a case where the compound A and the fulvestrant were administered.

OD-BRE-0438 is a hormone receptor-positive breast cancer patient-derived tumor established by Oncodesign SA. Subculture was performed by subcutaneously transplanting tumor pieces into the NOD-SCID mice. Each of the above-described tumors excised from the mice was chopped into about 5 mm squares, transplanted into the subcutaneous right side of each mouse using a trocar (Φ3.5 mm), and provided for evaluating the antitumor effect.

β-Estradiol (FUJIFILM Wako Pure Chemical Corporation) was made into a solution with 99.5% ethanol (FUJIFILM Wako Pure Chemical Corporation) at a concentration of 1 mg/mL, and then, the solution was prepared to a final concentration of 2.5 μg/mL using sterile water for water supply. This solution was administered to the mice in their drinking water from the day of tumor transplantation to the test end date.

The major axis and minor axis of each tumor was measured with an electronic digital caliper (Digimatic™ Caliper, Mitutoyo Corporation). The volume of each tumor was calculated according to the following equation.

Tumor volume(mm³)=longest diameter (mm)×short axis (mm)×short axis (mm)/2

The compound A was dissolved in purified water to a concentration of 2.5 mg/mL. As fulvestrant, a commercially available preparation (50 mg/mL) was used as it is.

30 days after tumor transplantation, the mice were assigned to each group so that the average tumor volume was the same.

The start day of administration was set to day 0, and the drug was administered under the following conditions.

The compound A was administered orally to the mice in each group at a dose of 25 mg/kg (10 mL/kg) once a day for 14 consecutive days. In addition, the fulvestrant was injected subcutaneously at a dose of 250 mg/kg (5 mL/kg) on days 0 and 7. A control group was left untreated.

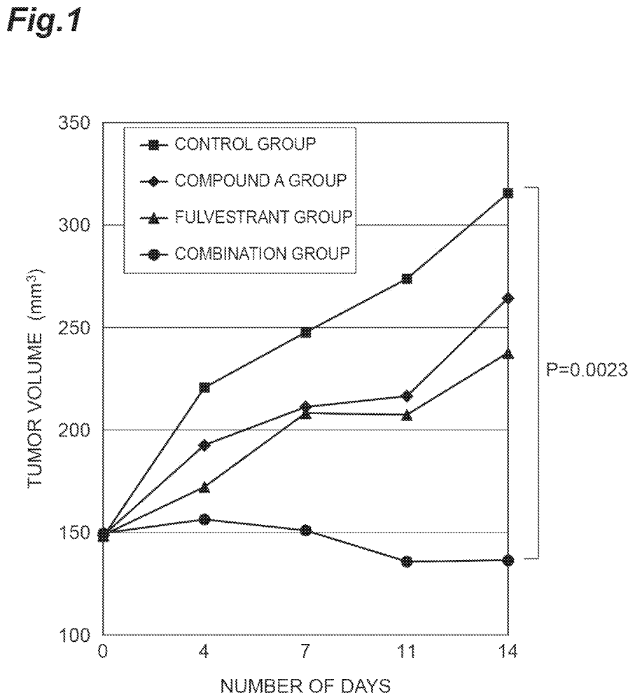

The tumor volume of each of the mice was measured on days 0, 4, 7, 11, and 14. The results (average values) are shown in Table 1 and FIG. 1. Dunnett's multiple test was performed on the tumor volume of the control group and each of the administration groups on day 14.

TABLE 1
Changes in average tumor volume after
starting drug administration (mm³)

| | Day 0 | Day 4 | Day 7 | Day 11 | Day 14 |
|---|---|---|---|---|---|
| Control | 147.9 | 220.7 | 247.6 | 273.7 | 315.5 |
| group (mm³) | | | | | |
| Compound | 148.6 | 192.6 | 211.2 | 216.5 | 264.3 |
| A 25 mg/kg | | | | | |
| group (mm³) | | | | | |
| Fulvestrant | 148.7 | 172.2 | 208.2 | 207.4 | 237.6 |
| 250 mg/kg | | | | | |
| group (mm³) | | | | | |
| Combination | 149.6 | 156.4 | 151.0 | 135.8 | 136.4 |
| group (mm³) | | | | | |

### Reference Example 1 Construction of Human CYP19A1 Expression Vector

A PB-CMV-MCS-EF1α-Puro vector (System Bioscience, LLC) was cleaved with Xba I and Not I, and a multiple cloning site was inserted thereinto to prepare a PB510B2 vector. Next, the PB510B2 vector was cleaved with Xba I and Cla I, and ORF of human CYP19A1 (NM_001347249.2) was inserted thereinto to obtain a PB510B2_hCYP19A1 vector.

### Reference Example 2 Construction of pC3-PBase Vector

A pC3-vector which was a vector obtained by removing a neomycin expression module (SV40 promoter-Neomycin ORF-SV40 poly A) of a pcDNA3 1(−) mammalian expression vector (Thermo Fisher Scientific Inc.) was constructed. Next, ORF of Super PiggyBac Transposase of Super PiggyBac Transposase Expression Vector (System Bioscience, LLC) was inserted into the downstream of CMV promoter of pC3-vector to obtain pC3-PBase vector.

### Reference Example 3 Establishment of Human-Derived Breast Cancer Cell Line ZR-75-1-hCYP19A1

A human-derived breast cancer cell line ZR-75-1 (ECACC) was seeded in a 6-well microplate (FALCON). RPMI-1640 medium (containing 4,500 mg/L glucose, L-glutamine, phenol red, HEPES, and sodium pyruvate, FUJIFILM Wako Pure Chemical Corporation) containing 10% FBS (SIGMA) and penicillin/streptomycin (FUJIFILM Wako Pure Chemical Corporation) was used as a medium. The seeded cells were cultured for 15 days under the conditions of 5% CO₂ and 37° C. using an incubator.

A liquid A which was obtained by mixing Lipofectamine™ 3000 reagent (Thermo Fisher Scientific Inc.) (3.75 μL) with Opti-MEM™ (Thermo Fisher Scientific Inc.) (125 μL) was prepared. In addition, the above prepared PB510B2_hCYP19A1 vector (2 μg), pC3-PBase (0.5 μg), P3000 reagent (Thermo Fisher Scientific Inc.)(5 μL), and Opti-MEM™ (125 μL) were mixed with each other to prepare a liquid B. These liquids A and B were mixed with each other, allowed to stand at room temperature for 5 minutes, and then added to the above-described ZR-75-1 cells obtained by culture, and the cells were cultured overnight under the conditions of 5% CO₂ and 37° C. Thereafter, culture was performed in a medium to which 1 μg/mL puromycin was added.

### Reference Example 4 Establishment of Human-Derived Breast Cancer Cell Line MCF-7-hCYP19A1

A human-derived breast cancer cell line MCF-7 (ECACC) was seeded in a 6-well microplate (FALCON). 10% FBS (SIGMA), penicillin/streptomycin (FUJIFILM Wako Pure Chemical Corporation), and E-MEM medium (containing L-glutamine, phenol red, sodium pyruvate, non-essential amino acids, and 1500 mg/L sodium hydrogen carbonate, FUJIFILM Wako Pure Chemical Corporation) were used as a medium. The seeded cells were cultured overnight under the conditions of 5% CO2 and 37° C. using an incubator.

A liquid A obtained by mixing Lipofectamine™ 3000 reagent (3.75 μL) with Opti-MEM™ (125 μL) was prepared. In addition, the above prepared PB510B2_hCYP19A1 vector (2 μg), pC3-PBase (0.5 μg), P3000 reagent (5 μL), and Opti-MEM™ (125 μL) were mixed with each other to prepare a liquid B. These liquids A and B were mixed with each other, allowed to stand at room temperature for 5 minutes, and then added to the above-described MCF-7 cells obtained by culture and the cells were cultured for 3 days under the conditions of 5% CO₂ and 37° C. Thereafter, the cells were cultured in a medium to which 1 μg/mL puromycin was added for 7 days, and then cultured in a medium to which 1.5 μg/mL puromycin was added.

### Example 2 Proliferation Inhibitory Action Against Subcutaneously Transplanted Tumor of Human-Derived Breast Cancer Cell Line ZR-75-1-hCYP19A1 Due to Combined Use of Compound a and Exemestane

Five BALB/c nude mice (CAnN.Cg-Foxn1/CrlCrlj, female, Charles River Laboratories Japan, Inc.) were used in each group to evaluate an antitumor effect in a case where the compound A and exemestane were administered.

The mice were subcutaneously injected with a mixed anesthetic solution of 0.3 mg/kg medetomidine hydrochloride, 4 mg/kg midazolam, and 5 mg/kg butorphanol tartrate. The skin and peritoneum on the back of each of the mice were incised, the left and right ovaries were resected, and the incision was sutured. Thereafter, 65 mg/kg (13.0 mg/mL, 100 μL/mouse) ampicillin sodium injection (Meiji Seika Pharma Co., Ltd.), 1 mg/kg (0.2 mg/mL, 100 μL/mouse) Antisedan (Nippon Zenyaku Kogyo Co., Ltd.), and 5 mg/kg (1.0 mg/mL, 100 μL/animal) carprofen injection (Zoetis Japan) were administered subcutaneously.

7 days later, androstenedione (Sigma-Aldrich) was administered orally to the mice at a dose of 0.1 mg/mouse (200 μL/mouse) until the test end date.

One day after the start of administration of androstenedione, 1×10⁷ cells/mouse of a human-derived breast cancer cell line ZR-75-1-hCYP19A1 was transplanted subcutaneously into the right sides of the mice. 20 days after the transplantation of the breast cancer cell line, the mice were assigned to each group so that the average tumor volume was the same.

The compound A was dissolved in purified water to a concentration of 2.5 mg/mL. In addition, Tween (registered trademark) 80 was mixed with a 0.5% methyl cellulose solution to a concentration of 0.4%. Exemestane was dissolved in this mixed solution to a concentration of 5 mg/mL.

The start day of administration was set to day 0, and the drug was administered under the following conditions. The compound A at a dose of 50 mg/kg (20 mL/kg), exemestane at a dose of 1 mg/mouse (200 μL/mouse), or the two agents of the compound A and exemestane (each at the same dose as described above), were administered orally to the mice in each group once a day for 11 consecutive days. A control group was left untreated.

The tumor volume of each of the mice was measured on days 0, 4, 7, and 11. The results (average values) are shown in Table 2. The major axis and minor axis of each tumor was measured with an electronic digital caliper (Digimatic™ Caliper, Mitutoyo Corporation). The volume of each tumor was calculated according to the following equation.

Tumor volume (mm³)=longest diameter (mm)×short axis (mm)×short axis (mm)/2

TABLE 2
Changes in average tumor volume after
starting drug administration (mm³)

| | Day 0 | Day 4 | Day 7 | Day 11 |
|---|---|---|---|---|
| Control group | 145.4 | 206.8 | 261.8 | 362.9 |
| (mm³) | | | | |
| Compound A | 147.3 | 187.7 | 232.8 | 319.9 |
| 50 mg/kg | | | | |
| group (mm³) | | | | |
| Exemestane 1 | 146.0 | 184.5 | 235.0 | 329.0 |
| mg/mouse | | | | |
| group (mm³) | | | | |
| Combination | 145.3 | 184.5 | 218.5 | 277.4 |
| group (mm³) | | | | |

### Example 3 Effect of Combining Compound a and Letrozole, Anastrozole, or Exemestane on Human-Derived Breast Cancer Cell Line MCF-7-hCYP19A1 in which Human Aromatase was Expressed

The human breast cancer cell line MCF-7-hCYP19A1 prepared in Reference Example 4 was cultured and maintained in a 5% CO₂ incubator using E-MEM medium (containing L-glutamine, phenol red, sodium pyruvate, non-essential amino acids, and 1500 mg/L sodium hydrogen carbonate, FUJIFILM Wako Pure Chemical Corporation) containing 10% FBS (SIGMA), penicillin/streptomycin (FUJIFILM Wako Pure Chemical Corporation), and 1.5 μg/mL puromycin (hereinafter referred to as a culture medium).

200 μL of each cell suspension prepared to 0.25×10⁴ cells/mL in the culture medium containing 10% FBS was added to each well of a cell culture plate and cultured overnight in the incubator.

On the next day, the medium was removed, and 100 μL of phenol red-free RPMI-1640 medium (FUJIFILM Wako Pure Chemical Corporation) containing 10% FBS (charcoal-stripped FBS, GIBCO), penicillin/streptomycin, and 1.5 μg/mL puromycin (hereinafter referred to as an assay medium) was added thereto, and culture was performed in the incubator.

On the next day, the medium was removed, 100 μL of an assay medium was added thereto to replace the medium, and the cells were cultured in the incubator for 2 days. Thereafter, the medium was removed, 25 μL of androstenedione prepared to a final concentration of 10 nmol/L in the assay medium and 25 μL of each of FGF2 (GIBCO) and FGF10 (R&D systems) respectively prepared to final concentrations of 25 ng/mL and 100 ng/mL in the assay medium were added thereto, and then, 25 μL of each solution described below was added thereto, and culture was performed in the incubator for 7 days. The medium was removed 3 days and 5 days after addition of the drugs, and 25 μL of each of the androstenedione, FGF2, and FGF10 solutions and each solution described below was added thereto to replace the medium.

(1) Assay medium containing letrozole (SIGMA), anastrozole (SIGMA), exemestane (SIGMA), or DMSO respectively prepared to final concentrations of 2,000 nmol/L, 10,000 or 2,000 nmol/L, 2,000 nmol/L or 400 nmol/L, and 0.05% in assay medium (used for control group or single agent of Compound A)

(2) Assay medium containing Compound A and DMSO respectively prepared to final concentrations of 1,000 nmol/L and 0.005% in assay medium (used for control group or single drug described in (1))

Using CellTiter Glo™ 2.0 (Promega), the number of cells in each of the wells after culture was calculated based on the amount of ATP contained in the cells obtained by measuring the emission intensity.

After removing the medium from the plate, 50 μL of a solution obtained by mixing the measurement reagent with the culture medium at 1:1 was added to each well. After mixing the plate in a plate mixer for 5 minutes, the emission was measured using a plate reader (EnVision™, PerkinElmer, Inc.). Average values (n=3) of each group for values obtained by subtracting emission values in cell-free wells from emission values of the wells are shown in Tables 3 to 7.

TABLE 3

| | Emission value (count |
|---|---|
| | per second) |
| Control group | 1,481,915 |
| Compound A 1,000 nmol/L | 1,242,509 |
| Letrozole 2,000 nmol/L | 1,410,920 |
| Combined use of Compound A and | 1,159,491 |
| letrozole | |

TABLE 4

| | Emission value (count |
|---|---|
| | per second) |
| Control group | 1,334,640 |
| Compound A 1,000 nmol/L | 1,157,051 |
| Anastrozole 10,000 nmol/L | 1,443,072 |
| Combined use of Compound A and | 924,395 |
| anastrozole | |

TABLE 5

| | Emission value (count |
|---|---|
| | per second) |
| Control group | 1,334,640 |
| Compound A 1,000 nmol/L | 1,157,051 |
| Anastrozole 2,000 nmol/L | 1,357,941 |
| Combined use of Compound A and | 1,018,781 |
| anastrozole | |

TABLE 6

| | Emission value (count |
|---|---|
| | per second) |
| Control group | 1,243,964 |
| Compound A 1,000 nmol/L | 1,164,308 |
| Exemestane 2,000 nmol/L | 1,244,423 |
| Combined use of Compound A and | 949,300 |
| exemestane | |

TABLE 7

| | Emission value (count |
|---|---|
| | per second) |
| Control group | 1,243,964 |
| Compound A 1,000 nmol/L | 1,164,308 |
| Exemestane 400 nmol/L | 1,324,919 |
| Combined use of Compound A and | 1,003,903 |
| exemestane | |

### Example 4 Proliferation Inhibitory Action Against Subcutaneously Transplanted Tumor of Human-Derived Breast Cancer Cell Line MCF-7-hCYP19A1 Due to Combined Use of Compound a and Letrozole

Five BALB/c nude mice (CAnN.Cg-Foxn1/CrlCrlj, female, Charles River Laboratories Japan, Inc.) were used in each group to evaluate an antitumor effect in a case where the compound A and the letrozole were administered.

The mice were subcutaneously injected with a mixed anesthetic solution of 0.3 mg/kg medetomidine hydrochloride, 4 mg/kg midazolam, and 5 mg/kg butorphanol tartrate. The skin and peritoneum on the back of each of the mice were incised, the left and right ovaries were resected, and the incision was sutured. Thereafter, Baytril 2.5% injection (Bayer) diluted with physiological saline (Otsuka Pharmaceutical Factory, Inc.) to a final concentration of 1 mg/mL as enrofloxacin; and antisedan (Nippon Zenyaku Kogyo Co., Ltd.) diluted with physiological saline (Otsuka Pharmaceutical Factory, Inc.) to a final concentration of 0.2 mg/mL as atipamezole hydrochloride were administered subcutaneously at respective amounts equivalent to 5 mg/kg (100 μL/mouse) and 1 mg/kg (100 μL/mouse).

7 days later, a solution obtained by diluting testosterone enanthate intramuscular injection (Fuji Pharma Co., Ltd.) with sesame oil to a concentration of 15.6 mg/mL was administered intramuscularly at a concentration of 0.1 mL/mouse (1.56 mg/mouse). Thereafter, the solution was administered intramuscularly to the mice once a week until the test end date.

One day after the start of administration of testosterone enanthate, 1×10⁷ cells/mouse of the human-derived breast cancer cell line MCF-7-hCYP19A1 was transplanted subcutaneously into the right sides of the mice. 9 days after transplantation of the breast cancer cell line, the mice were assigned to each group so that the average tumor volume was the same.

The compound A was dissolved in purified water to a concentration of 2.5 mg/mL. In addition, letrozole was dissolved in sterilized 0.3% hydroxypropyl cellulose water to a concentration of 0.05 mg/mL.

The start day of administration was set to day 0, and the drug was administered under the following conditions. The compound A at a dose of 25 mg/kg (10 mL/kg), letrozole at a dose of 0.01 mg/mouse (200 μL/mouse), or the two agents of the compound A and the letrozole (each at the same dose as described above), were administered orally to the mice in each group once a day for 14 consecutive days. A control group was left untreated.

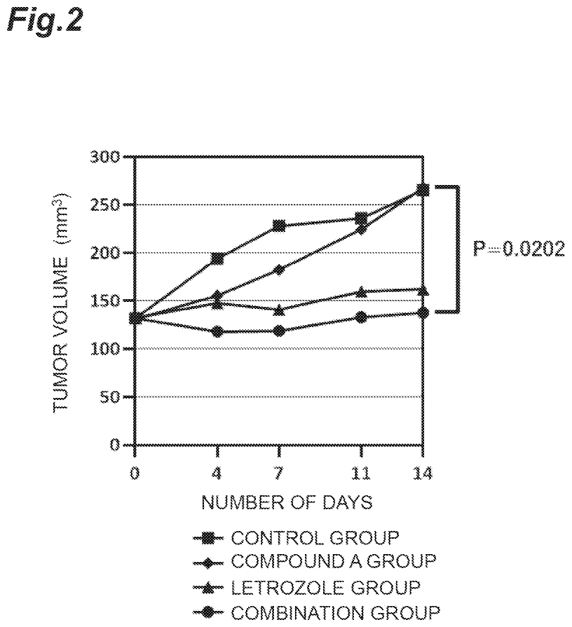

The tumor volume of each of the mice was measured on days 0, 4, 7, 11, and 14. The results (average values) are shown in Table 8 and FIG. 2. The major axis and minor axis of each tumor was measured with an electronic digital caliper (Digimatic™ Caliper, Mitutoyo Corporation). The volume of each tumor was calculated according to the following equation.

Tumor volume(mm³)=longest diameter (mm)×short axis (mm)×short axis (mm)/2

Dunnett's multiple test was performed on the tumor volume of the control group and each of the administration groups on day 14.

TABLE 8
Changes in average tumor volume after
starting drug administration (mm³)

| | Day 0 | Day 4 | Day 7 | Day 11 | Day 14 |
|---|---|---|---|---|---|
| Control group (mm³) | 131.7 | 193.9 | 227.8 | 235.9 | 265.2 |
| Compound A | 130.7 | 155.5 | 182.4 | 224.4 | 267.5 |
| 25 mg/kg group (mm³) | | | | | |
| Letrozole | 132.4 | 147.3 | 140.6 | 159.6 | 162.0 |
| 0.01 mg/mouse group | | | | | |
| (mm³) | | | | | |
| Combination group | 132.0 | 117.9 | 118.6 | 132.9 | 137.3 |
| (mm³) | | | | | |

1-19. (canceled)

20. A method for treating breast cancer, comprising:
administering 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I): 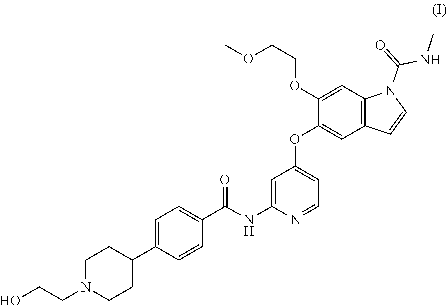 [SMILES: CNC(=O)n1ccc2cc(Oc3ccnc(NC(=O)c4ccc(C5CCN(CCO)CC5)cc4)c3)c(OCCOC)cc21]
or a pharmacologically acceptable salt thereof, and an estrogen receptor antagonist or an aromatase inhibitor to a patient in need thereof.

21. The method according to claim 20,
wherein 5-((2-(4-(1-(2-hydroxyethyl)piperidin-4-yl)benzamide)pyridin-4-yl)oxy)-6-(2-methoxyethoxy)-N-methyl-1H-indole-1-carboxamide represented by Formula (I) or a pharmacologically acceptable salt thereof; and the estrogen receptor antagonist or the aromatase inhibitor are administered simultaneously or separately.

22. The method according to claim 20, wherein the pharmacologically acceptable salt is 1.5 succinate.

23. The method according to claim 20, wherein the estrogen receptor antagonist is fulvestrant, tamoxifen or a pharmacologically acceptable salt thereof, or mepitiostane.

24. The method according to claim 20, wherein the estrogen receptor antagonist is fulvestrant.

25. The method according to claim 20, wherein the aromatase inhibitor is exemestane, anastrozole, or letrozole.

26. The method according to claim 20, wherein the aromatase inhibitor is exemestane.

27. The method according to claim 20, wherein the aromatase inhibitor is letrozole.

28. The method according to claim 20, wherein the breast cancer is estrogen receptor-positive.

29. The method according to claim 20, wherein the breast cancer is locally advanced breast cancer, metastatic breast cancer, recurrent breast cancer, or unresectable breast cancer.

30. The method according to claim 20, which is for use in treatment of breast cancer expressing fibroblast growth factor receptor (FGFR).

31. The method according to claim 30, wherein FGFR is FGFR1, FGFR2, or FGFR3.

32. A pharmaceutical composition for treating breast cancer, comprising:
5-((2-(4-(1-(2-hydroxyethyl)piperidin-4-yl)benzamide)pyridin-4-yl)oxy)-6-(2-methoxyethoxy)-N-methyl-1H-indole-1-carboxamide or a pharmacologically acceptable salt thereof; and an estrogen receptor antagonist or an aromatase inhibitor.

33. The pharmaceutical composition according to claim 32, wherein the pharmacologically acceptable salt is 1.5 succinate.

34. The pharmaceutical composition according to claim 32, wherein the estrogen receptor antagonist is fulvestrant, tamoxifen or a pharmacologically acceptable salt thereof, or mepitiostane.

35. The pharmaceutical composition according to claim 32, wherein the estrogen receptor antagonist is fulvestrant.

36. The pharmaceutical composition according to claim 32, wherein the aromatase inhibitor is exemestane, anastrozole, or letrozole.

37. The pharmaceutical composition according to claim 32, wherein the aromatase inhibitor is exemestane.

38. The pharmaceutical composition according to claim 32, wherein the aromatase inhibitor is letrozole.
